(19) United States
(12) Plant Patent Application Publication
(10) Pub. No.: US 2012/0151645 P1
(43) Pub. Date: Jun. 14, 2012

(54) Blackberry Plant Named 'Reuben'

(50) ;

(76) Inventors: John Reuben Clark, Fayetteville, AR (US); Jane Fairlie, Spalding (GB)

(21) Appl. No.: 12/928,424

(22) Filed: Dec. 10, 2010

(51) Int. Cl. A01H 5/00

(52) U.S. Cl. PLT/203

(57) ABSTRACT

This invention relates to a new and distinct variety of blackberry plant named ‘Reuben’, which is primarily characterized by its primocane fruiting habit, large fruit size, high quality, and high productivity, is disclosed.

### GENUS AND SPECIES

Rubus subgenus Eubatus sect. Moriferi & Ursini

### VARIETY DENOMINATION

‘Reuben’

### BACKGROUND OF THE NEW PLANT

The present invention relates to a new and distinct blackberry variety designated ‘Reuben’ and botanically known as Rubus subgenus Eubatus sect. Moriferi & Ursini. This new Blackberry variety was discovered in 2005 in Fayetteville, Ark., and originated from a hand pollinated cross between the female parent blackberry plant ‘A-2292T’ (unpatented) and the male parent blackberry plant ‘APF-44’ (unpatented). Seeds from this cross were shipped to Spalding, Lincs, United Kingdom, in November of 2005, where the seeds were germinated. In summer 2006, the seeds were field planted and, in late autumn 2006, one seedling, designated HPB3, was selected for its primocane fruiting habit, large fruit size, high quality, and high productivity.

The new variety ‘Reuben’ was first propagated via root cuttings and was subsequently propagated via root cuttings in February 2009 in Spalding, Lincs, United Kingdom. ‘Reuben’ has been asexually reproduced by root cuttings and in-vitro propagation for 2 generations. The present invention has been found to be stable and reproduce true to type through successive asexual propagations via root cuttings.

### PLANT BREEDER'S RIGHTS

Plant Breeder's Rights for this variety were applied for in the European Union on Dec. 14, 2009. ‘Reuben’ has not been made sold or made publicly available more than one year prior to the filing date of this application.

### SUMMARY OF THE INVENTION

The following are the most outstanding and distinguishing characteristics of this new variety when grown under normal horticultural practices in Spalding, Lincs, United Kingdom.
1. A primocane fruiting habit;
2. Large fruit size;
3. High quality; and
4. High productivity.

### DESCRIPTION OF THE PHOTOGRAPHS

This new Blackberry plant is illustrated by the accompanying photographs, which show the fruit of the plant as well as the canes. The colors shown are as true as can be reasonably obtained by conventional photographic procedures. The photographs are of two-year-old plants.

FIG. 1 shows the whole plant, including foliage and flowers.

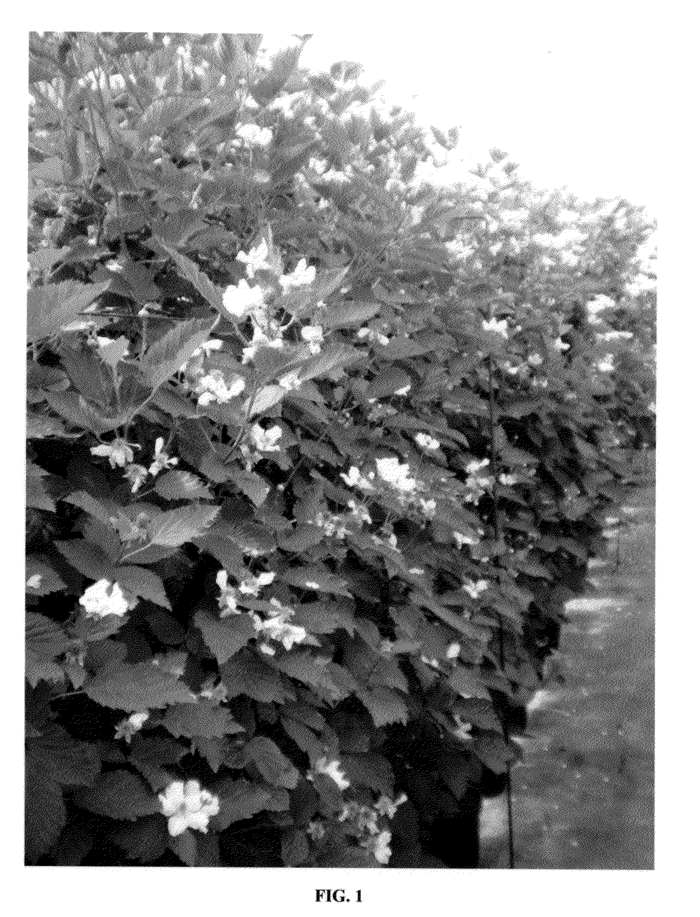

FIG. 2 shows a close-up of the flowers.

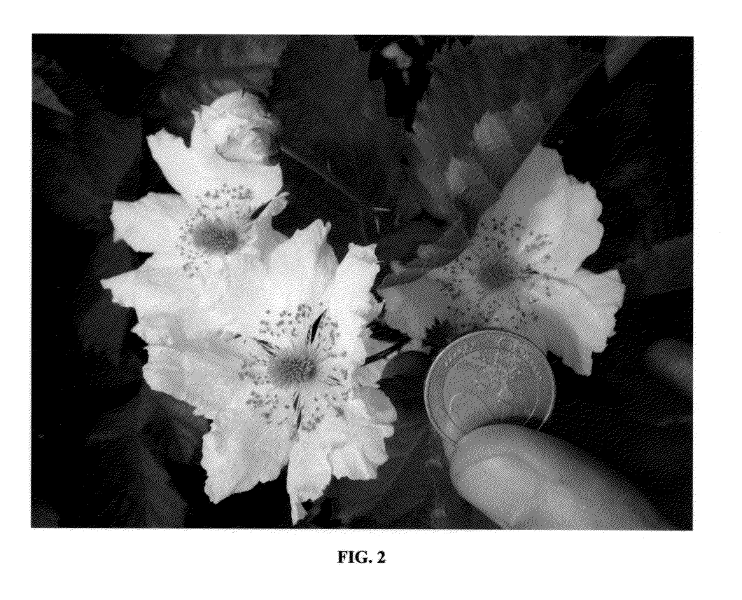

FIG. 3 shows a cane with both immature and mature berries.

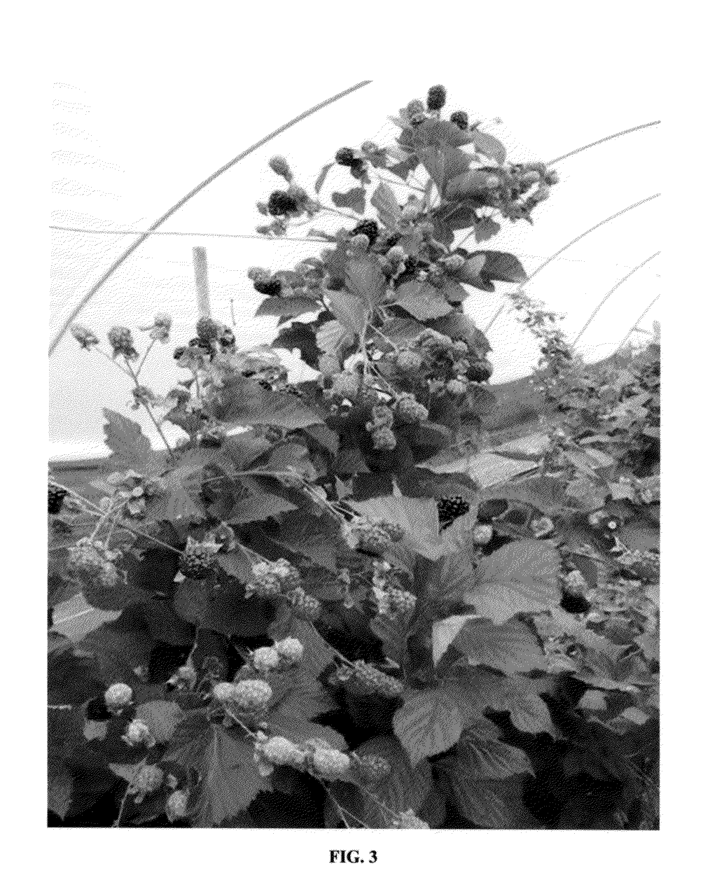

FIG. 4 shows a close-up of both immature and mature berries.

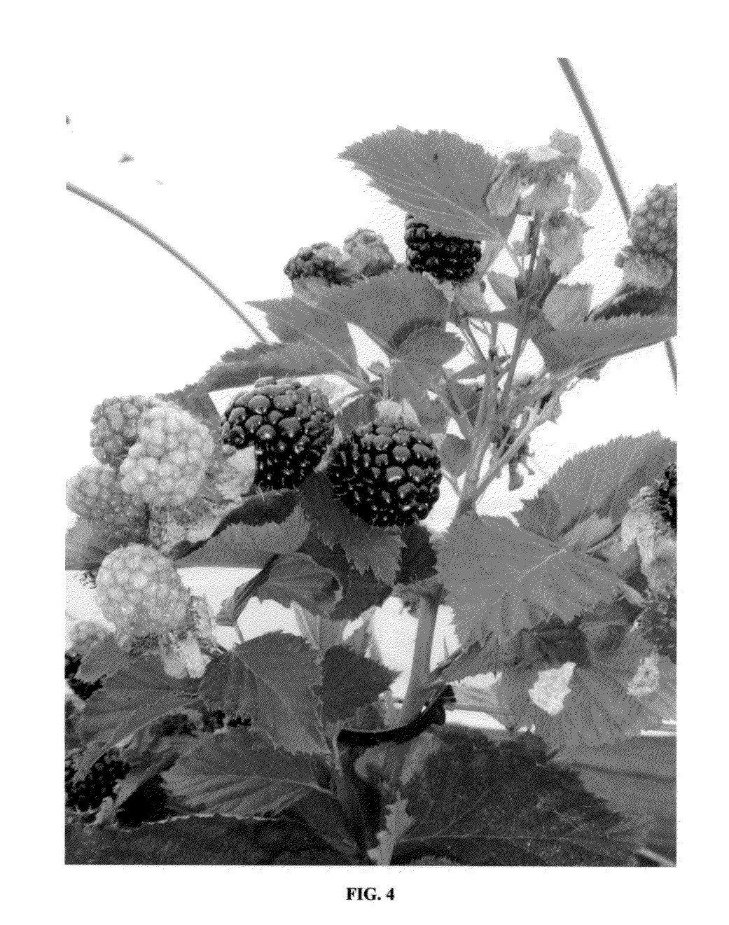

### DESCRIPTION OF THE NEW VARIETY

The following description of ‘Reuben’ is based on observations of two-year-old plants taken from 2006 to 2008 in Spalding, Lincs, United Kingdom. Description is in accordance with UPOV terminology. ‘Reuben’ has not been observed under all possible environmental conditions. Color terminology follows The Royal Horticultural Society Colour Chart, London (R.H.S.) (2001 edition).

### DETAILED BOTANICAL DESCRIPTION

Classification:
   Family.—Rosaceae.
   Botanical.—Rubus subgenus Eubatus sect. Moriferi & Ursini.
   Common name.—Blackberry.
   Variety name.—‘Reuben’.
Parentage:
   Female parent.—The blackberry plant ‘A-2292T’ (unpatented).
   Male parent.—The blackberry plant ‘APF-44’ (unpatented).
Plant:
   Vigor.—Medium.
   Growth habit.—Erect and compact.
   Productivity.—High.
   Propagation.—Tissue culture and root cuttings.
   Self-fruitfulness.—Self-fertile.
Fruiting:
   Lateral length.—Medium.
   Length (4^{th} lateral from tip) average (cm).—7.5 cm.
   Number of fruit per lateral.—Average: 10 Range: 8 to 12.
Shoot:
   Number of young shoots.—6 to 8 per plant.
   Very young shoots intensity of anthocyanin coloration.—Weak.
   Time of young shoot emergence from soil (under normal conditions in the United Kingdom).—April.
   Young shoot pigmentation.—Medium-green.
   Shoot length (cm).—Average: 200 Range: 150 cm to 250 cm.
   Glaucosity (waxy bloom) (observed on full-grown shoot after picking).—Present.
   Cane cross section (from mid cane observed at end of first growing season).—Rounded.
   Dormant cane color.—Pale-brown.
   Pubescence on canes.—Few, downward.
Cane internodal distance (distance between nodes):
   Internodal distance (cm) (at central ⅓ of cane).—Average: 6.0 cm Range: 3.0 cm to 7.0 cm.
Leaves:
   Color.—Upper surface: RHS N137A Lower surface: RHS N137D.
   Arrangement.—Palmate.
   Number of leaflets.—5.
   Relief between veins.—Weak.
   Glossiness.—Medium.
   Number of leaflets.—5.
   Cross section profile.—V-shape.
   Petiole.—Length (cm): Average: 3.0 cm Range: 3.0 cm to 6.0 cm Pigmentation of upper surface: Pale green Pigmentation color of upper surface: RHS 143C Pigmentation of lower surface: Pale green Pigmentation color of lower surface: RHS 143C.
   Terminal leaflet.—Length: Average: 11.0 cm Range: 9.0 cm to 14.5 cm Width: Average: 9.5 cm Range: 7.5 cm to 11.5 cm.
Inflorescence:
   Flower size.—Large.
   Flower diameter.—Average: 6.1 cm Range: 4.0 cm to 7.1 cm.
   Diameter of calyx relative to corolla.—2.1 cm.
   Flower number (at 3^{rd} node from tip of lateral).—Average: 5 Range: 4 to 8.
   Petal.—Length: Average: 2.4 cm Range: 2.1 cm to 2.4 cm Width: Average: 1.7 cm Range: 1.4 cm to 1.9 cm Color: White Shape: Oval Margin: 2.4 Length/width ratio (observe on secondary flowers): 1.4:1.0 cm.
   Sepal.—Color: RHS 147C.
   Pedicel.—Length: 0.9 cm.
Fruit:
   Time of ripening.—Late.
   Harvest interval.—August to November.
   Length of harvest season.—10 to 12 weeks.
   Color.—Immature: RHS 203A Maturing: RHS 203B Mature: RHS 203C.
   Glossiness.—Strong.
   Shape.—Oblong, blocky.
   Length.—Average: 2.7 cm Range: 2.5 cm to 2.9 cm.
   Width.—Average: 2.1 cm Range: 1.8 cm to 2.3 cm.
   Ratio of length to width.—1.28:1.0 cm.
   Weight (g/fruit).—14.5.
   Soluble solids (%) (in Brix).—11.5%.
   Number of druplets/fruit.—45.
   Firmness.—Very firm.
   Yield.—3.5 kg of fruit per plant.

### COMPARISON WITH PARENTAL AND COMMERCIAL VARIETIES

‘Reuben’ differs from the female parent ‘A-2292T’ (unpatented) in that ‘Reuben’ is primocane fruiting, while ‘A-2292T’ is floricane fruiting. Additionally, ‘Reuben’ is larger fruited and has a fruit that weighs on average 14.5 g, while fruit of ‘A-2292T’ weighs on average 6.0 g. Furthermore, ‘Reuben’ is thorny, while ‘A-2292T’ is thornless.

‘Reuben’ differs from the male parent ‘APF-44’ (unpatented) in that ‘Reuben’ is more vigorous and taller in primocane height than ‘APF-44’. Additionally, ‘Reuben’ has larger fruit size potential, with a fruit that weighs on average 14.5 g, while fruit of ‘APF-44’ weighs on average 8.0 g.

‘Reuben’ differs from the commercial variety blackberry plant named ‘Natchez’ (U.S. Plant Pat. No. 20,891) in that ‘Reuben’ has larger fruit that weighs on average 14.5 g, while the fruit of ‘Natchez’ averages 8.9 g to 9.4 g. In addition, the plants of ‘Reuben’ are thorny, while the plants of ‘Natchez’ are thornless.

I claim:

1. A new and distinct variety of blackberry plant as described and shown herein.
